(19) United States
(12) Patent Application Publication
(10) Pub. No.: US 2004/0059190 A1
(43) Pub. Date: Mar. 25, 2004

(54) Method of colpoplasty

(76) Inventor: David L. Matlock, Los Angeles, CA (US)

Correspondence Address: PERKINS COIE LLP, POST OFFICE BOX 1208, SEATTLE, WA, 98111-1208, US

(21) Appl. No.: 10/461,894

(22) Filed: Jun. 13, 2003

Related U.S. Application Data
(60) Provisional application No. 60/394,947, filed on Jul. 8, 2002.

(51) Int. Cl. A61F 5/00

(52) U.S. Cl. 600/38

(57) ABSTRACT

Disclosed is a method of colpoplasty wherein the colpoplasty is directed to augmentation, either temporary of permanent, of the Grafenberg Spot area. The invention disclosed is beneficial in the treatment of female sexual disorders and is beneficial for women with normal sexual response and esthesia. Also disclosed are kits for performing permanent colpoplastic augmentation of the Grafenberg area. One preferred embodiment of a temporary colpoplasty uses injectable collagen obtained from a tissue bank. One preferred embodiment of a permanent colpoplasty uses implantable ePTFE.

This application claims the benefit of U.S. Provisional Application No. 60/394,947 filed Jul. 8, 2002 and which is incorporated by reference as if fully set forth herein.

### FIELD OF THE INVENTION

The following disclosure relates to the field of gynecology, vaginal augmentation, normal and abnormal female sexual response and female sexual dysfunction (FSD).

### BACKGROUND OF THE INVENTION

This invention relates generally to the medical field of female sexual response and esthesia via vaginal augmentation, or colpoplasty. Primarily, normal female sexual response, female sexual disorders, esthesia, dysesthesia and dyserethism are considered. Historically, clinical and academic medicine have been focused primarily on the male sexual response and understanding male sexuality. Attitudes and research of normal and abnormal female sexual response and sexuality have lagged behind the advancements and understandings relating to men. In the past, the female sex drive was seen as connected to the purpose of procreation only. Since the publication of the best selling “The Hite Report: A Nationwide Study on Female,” open discussion and intensified research relating to female sexuality increased.

In research and discussion relating to female sexuality, FSD has been described and found to be age-related, progressive and highly prevalent, affecting 30-50 percent of women. Rosen R C, Taylor J F, Leiblum S R, et al: Prevalence of sexual dysfunction in women: results of a survey study of 329 women in an outpatient gynecological clinic. J. Sex. Mar. Ther. 19:171-188 (1993); Read S, King M, Watson J: Sexual dysfunction in primary medical care: prevalence, characteristics and detection by the general practitioner. J. Public Health Med. 19:387-391 (1997). In the National Health and Social Life Survey of 1,749 women, 43 percent experienced sexual dysfunction. Laumann E, Paik A, Rosen R., Sexual Dysfunction in the United States Prevalence and Predictors. JAMA 281: 537-544 (1999). U.S. population census data revealed that 9.7 million American women ages 50-74 self-reported complaints of diminished vaginal lubrication, pain and discomfort with intercourse, decreased arousal, and difficulty achieving orgasm. Female sexual dysfunction is an important women's health issue that affects the quality of life of many women across the world. Id.

In all, there has been little research or attention that focused on female sexual function. Knowledge and understanding of the anatomy and physiology of the female sexual response has been, and still is, quite limited. Based on an understanding of the physiology of the male erectile response, recent advances in modern technology, including the commonly prescribed sildenafil, and recent interest in women's health issues, the study of female sexual dysfunction has gradually evolved. Study in this area has led to findings that FSD includes a multitude of sexual disorders.

In other research, principally in 1981, the Grafenberg Spot was described by John Perry and Beverly Whipple and published in the Journal of Sex Research. They reported an area that formed a neural reflex loop from the female genitalia that could result in orgasm and that was independent of the previously described clitoral reflex loop. Perry and Whipple reported that they had had a physician or nurse examine more than 400 women who had volunteered to be research subjects, and a hyperesthetic, or increased sensitivity, area in the vagina was found in each of these women. They cautioned, however, that they could not state with certainty that every woman had such a sensitive area. They further reported that a second reflex pathway included the Grafenberg spot as the major source of stimulation, the pelvic nerve and hypogastric plexus as its major pathway and the musculature of the uterus, bladder, urethra, contractile elements associated with paraurethral glands and the proximal portion of the pubococcygeus muscle as its major myotonic responders. This double reflex system, the vulvar and uterine reflex systems, were accepted over time. See, <http://www.hisandherhealth.com/articles/>.

The Grafenberg spot has been theorized to have adaptive significance in that studies on pain thresholds revealed that adequate pressure to the Grafenberg area could result in a pain blocking effect equal to or greater than ten mg of morphine per kilogram of body weight. This vaginocervical pain blocking mechanism is believed to play a part in a woman's tolerance to the pain endured in labor and parturition. Id.

The Grafenberg spot was named by John Perry and Beverly Whipple to commemorate the research of Ernst Grafenberg, a German-born obstetrician and gynecologist, who in 1944, along with Robert L. Dickinson, described a zone of heightened sensation and erogenous feeling located along the suburethral surface of the anterior vaginal wall. In 1950, Grafenberg wrote that a zone of heightened sensitivity could always be demonstrated on the anterior wall of the vagina along the course of the urethra, which seemed to be surrounded by erectile tissue, similar to the corpora cavernosa of the penis. In an anonymous questionnaire distributed to professional women in the United States and Canada, Davidson and colleagues found that 786 (66 percent) of 1,245 respondents perceived an especially sensitive area in their vagina that, if stimulated, produced pleasurable feelings. Whipple found that of the 800 women who completed a sexual-health questionnaire, 69 percent of the subjects reported the mid-sagital line between the neck of the bladder and the pubic symphysis were the most sensitive area.

It is at least one object of this invention to offer treatment to what is now understood as a vast array of medical and psychological conditions resulting in FSD. Further, it is an object of this invention to positively augment normal sex lives of women, who do not suffer FSD, and their partners.

### SUMMARY OF THE INVENTION

This disclosure is directed to both normally functioning sexually active women and those women suffering from FSD. Specifically, the invention is directed to a colpoplasty involving the anterior wall of the vagina, Grafenberg spot (GS) area, and the augmentation thereof. At the GS area an injection of collagen or other appropriate substance is placed in the submucosal layer deep to the GS area, distal to the bladder and proximal to the distal aspect of the urethra.

In another embodiment of the invention, a colpoplasty is described wherein the implantation by way of injection or otherwise is temporary or permanent.

In another embodiment of the invention, a kit is disclosed that can be used to perform colpoplasty to augment the GS area. In the above embodiments, an implantation, injection or otherwise, is of an inert compound which is implanted deep to, or anterior to the mucosal layer of the vaginal wall at the midline of the body at a location posterior to the pubic symphysis. The substance injected or implanted otherwise may be permanent, i.e., non-absorbable or absorbable.

It is important to note that in each embodiment of the invention the exact anatomical location of the GS area identified for colpoplasty will be subject dependent. To ameliorate the difficulty involved in proper location of the GS area and the individualized anatomical location thereof, a speculum primarily designed for colpoplastic augmentation of the GS area is disclosed herein. Speculums are well known in the art.

### BRIEF DESCRIPTION OF THE FIGURES

FIG. 1 illustrates the esthesia in post colpoplastic augmentation of the GS area by collagen injection. Results are expressed as a percentage of pre-injection and post-injection surveys, and are presented as means +1-SEM (error bars) from 15 women who participated in the study. *p<0.05.

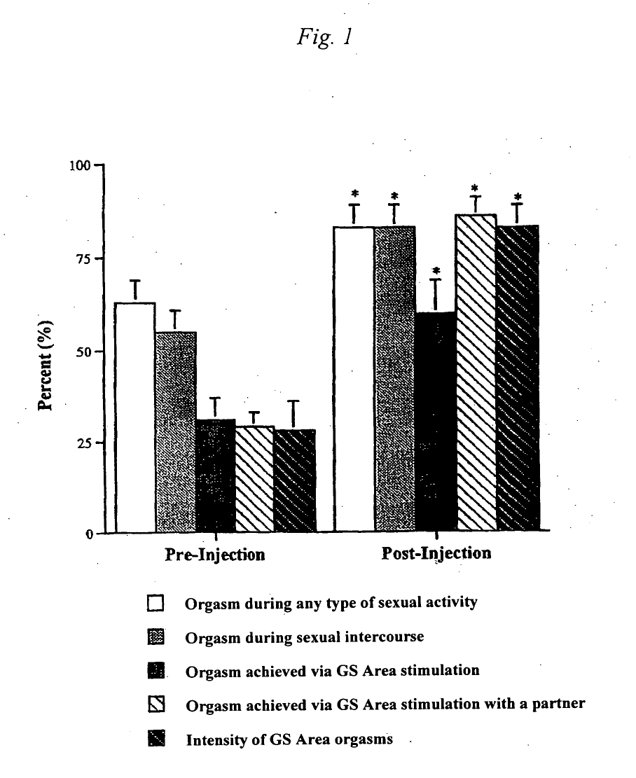

### DEFINITIONS

For the disclosure herein:

The term “absorbable material” refers to a material that will be reabsorbed by the subject's body within a period of 4-6 months.

The term “anorgasmic” means a persistent or recurrent difficulty, delay in, or absence of attaining orgasm following sufficient sexual stimulation and arousal.

The term “chemotherapy” is used as a broad term to describe any therapy based on prescribing or administering pharmaceuticals. In this respect, chemotherapy includes the simple taking of aspirin to the more difficult alteration of hormonal body chemistry. Also included are aphrodisiacs and amino acids used for treatment of FSD.

The term “climax” means orgasm whether by vulvar, vaginal, cervical or clitoral stimulation.

The term “colpoplasty” refers to the injection of absorbable or non-absorbable materials or implantation of absorbable or non-absorbable materials deep to an identified or assumed GS area about the vaginal submucosa. Colpoplasty also refers to an augmentation of the GS area consistent with the invention disclosed herein.

“Deep” is an anatomical term used to denote one structure or area as related to a more superficial area. For example, an area deep to the spine would be plural cavity. An area deep to the umbilicus would be the lower alimentary canal. Because the vagina subtends an anatomical void, an area deep to the anterior wall of the vagina would be structures that are more anterior whereas an area deep to the posterior wall would be more posterior and an area deep to the cervix would be more superior.

“GS area” as used herein, means the area about Grafenberg spot, including the Grafenberg spot, that is consistent with findings of erectile tissue along the course of the urethra which help complete a neural reflex loop, which may include nerve fibers from the hypogastric plexus and the pelvic nerve and in which stimulation may result in climax apart from or including clitoral stimulation. It is understood that the anatomy of the GS area will be subject specific and may occupy varying locations and sizes within a population. The “GS proper” as used herein, is the traditionally understood Grafenberg Spot.

The term “hypolubrication,” as used herein means an inability of a female to produce adequate vaginal lubrication via organic processes.

The term psychotherapy is interchangeable with psychology or psychiatry and includes family therapy, sex counseling, marriage counseling and relationship counseling.

The term “removably attachable light source” refers to a light source that may be connected or attached to a speculum or used without being attached. The light source may be attached or removed without negatively disrupting colpoplastic or other vaginal procedures.

### DETAILED DESCRIPTION OF THE INVENTION

FSD may include a multitude of psychological and medical symptoms. It is contemplated that women with these disorders, as well as normal functioning women, will benefit from the disclosed procedure. In Hypoactive Sexual Desire Disorder (HSDD) woman may complain of persistent or recurring deficiency (or absence) of sexual fantasies/thoughts, and/or receptivity to sexual activity, which causes personal and relationship distress. In Sexual Aversion Disorder (SAD) a woman may present with persistent or recurring phobic aversion to and avoidance of sexual contact with a sexual partner, which causes personal and relationship distress. SAD is generally a psychologically or emotionally based problem that can result for a variety of reasons such as physical or sexual abuse, or childhood trauma, etc.

HSDD may result from psychological/emotional factors or be secondary to medical problems such as hormone deficiencies and medical or surgical interventions. Any disruption of the female hormonal system caused by natural menopause, surgically or medically induced menopause or endocrine disorders can result in inhibited sexual desire.

Other disorders of arousal include, but are not limited to, lack of or diminished vaginal lubrication, decreased clitoral and labial sensation, decreased clitoral and labial engorgement or lack of vaginal smooth muscle relaxation. These conditions may occur secondary to psychological factors however, often there is a medical/physiologic basis such as diminished vaginal/clitoral blood flow, prior pelvic trauma, pelvic surgery, and various medications. In many of these cases, simple GS augmentation alone may be of benefit, and may prove additionally useful in combined psychological and or medical treatments.

Anorgasmic Disorder is a persistent or recurrent difficulty, delay in, or absence of attaining orgasm following sufficient sexual stimulation and arousal, and causes personal distress and potential relationship problems. This may be a primary (never achieved orgasm) or secondary condition, as a result of surgery, trauma, posttraumatic stress disorder or hormone deficiencies. Primary anorgasmia can be caused to emotional trauma or sexual abuse, however medical/physical factors commonly contribute to the problem.

Sexual Pain Disorders (SPD) have been described and contain: dyspareunia, a recurrent or persistent genital pain associated with sexual intercourse; vaginismus, a recurrent or persistent involuntary spasm of the musculature of the outer third of the vagina that interferes with vaginal penetration, which causes personal distress; and recurrent or persistent genital pain induced by non-coital sexual stimulation. Vaginismus usually develops as a conditioned response to painful penetration, or secondary to psychological/emotional factors. Further, this condition may be the factitious or based upon conversion reaction. Dyspareunia can develop secondary to medical problems such as vestibulitis, vaginal atrophy, or vaginal infection and can be either physiologically or psychologically based or a combination of the two.

The above conditions may benefit from combined therapy, i.e. chemotherapy with colpoplasty, psychology with colopoplasty, etc.

It is well known that hormones play a significant role in regulating female sexual function and response. In animal models, estrogen administration results in expanded touch receptor zones, which suggests that estrogen effects esthesia. In post-menopausal women, estrogen therapy helps to restore clitoral and vaginal vibratory sensation, two point discrimination sensation, sharp/dull and hot/cold sensation as well as proprioception. It has been reported that hormone treatments may increase sensation levels comparable to pre-menopausal women. Sarrel P M, Sexuality and Menopause. Obstet/Gynecol. 75: 26-30 (1990). Further, estrogens have protective effects that result in increased blood flow to the vagina and clitoris. Sarrel P M., Sexuality and Menopause. Obstet/Gynecol. 75: 26s-30s (1990); Sarrel P M., Ovarian hormones and vaginal blood flow: using laser Doppler velocimetry to measure effects in a clinical trial of post-menopausal women. Int. J. Impot. Rs. 10: 91-93 (1998). Over time, chemotherapy in this respect furthers the maintenance of FSD female's sexual response and esthesia.

It is known that with aging and menopause, and with the associated decreasing estrogen levels,. a majority of women experience some degree of change in sexual function. Specifically, some of the common sexual complaints include loss of desire, decreased frequency of sexual activity, painful intercourse, diminished sexual responsiveness, difficulty achieving orgasm, and decreased genital sensation. Ultimately, these changes may lead to marked dissatisfaction and relationship issues.

Masters and Johnson first published their findings of the physical changes occurring in menopausal women that related to sexual function in 1966. It has since been discovered that symptoms of hypolubrication and neuropathy are in part secondary to declining estrogen levels and that there is a direct correlation between the presence of sexual complaints and low levels of estrogen. These findings support conclusions directed to treating certain FSD symptoms with chemotherapy. Sarrel P M, Sexuality and Menopause. Obstet/Gynecol. 75: 26-30 (1990). Sarrel noted that symptoms markedly improve with estrogen replacement. It follows that enhancement of esthesia via colpoplasty in women undergoing estrogen replacement therapy will further diminish the symptoms of FSD cased by low estrogen levels.

Low testosterone levels are also associated with a decline in sexual arousal, genital sensation, libido, and orgasm. Multiple studies have documented and confirmed improvements in women's desire when treated with therapeutic doses of testosterone. Berman J, McCarthy M, Kyprianou N, Effect of estrogen withdrawal on nitric oxide synthase expression and apoptosis in the rat vagina. Urology 44: 650-656 (1998); Burger H G, Hailes J, Menelaus M, et al., The management of persistent menopausal symptoms with estradiol-testosterone implants. Maturitas 6: 35 (1984).

Additional causes of FSD include, but are limited to, vascular insufficiency, hypertension, elevated cholesterol levels, diabetes mellitus with pudendal, hypogastric and/or pelvic nerve neuropathy, smoking, and heart disease. Any traumatic injury to the genitals or pelvic region, such as pelvic fractures, blunt trauma and surgical disruption can result in diminished vaginal and clitoral blood flow and complaints of FSD.

The same neurological disorders that cause erectile dysfunction (ED) in men can also cause sexual dysfunction in women. Diabetes, myasthenia gravis, alcoholism, syrigomyelia, multiple sclerosis, etc., are included. Spinal cord injury or disease of the central or peripheral nervous system can result in FSD. As expected, women with spinal cord injury have significantly more difficulty achieving orgasm than able-bodied women. Tarcan T, Park K, Goldstein I, et.al., Histomorphometric analysis of age-related structural changes in human clitoral cavernosal tissue. J. Urol. (1999).

Dysfunction of the hypothalamic/pituitary axis, iatrogenic causes, surgical or medical castration, natural menopause, premature ovarian failure, and long term use of birth control pills, are the most common causes of hormonally based FSD.

Psychological factors are involved in nearly all cases of FSD. Despite the presence or absence of organic disease, emotional and relational issues significantly effect sexual arousal. Issues such as self-esteem, body image, relationships with sexual partners and the ability to communicate sexual needs all impact sexual function. In addition, psychological disorders such as depression, obsessive compulsive disorder, anxiety disorder, etc., are associated with FSD. Unfortunately, many medications used to treat depression can also significantly effect the female sexual response. The most frequently used medications for uncomplicated depression are the Serum Seratonin Re-uptake Inhibitors (SSRI). Women receiving these medications often complain of decreased sexual interest, inability to climax and hypolubrication.

Current treatment of FSD is gradually evolving as more clinical and basic scientific studies are dedicated to evaluating the problem and its processes. However, nearly all treatments need to be individualized and many carry concomitant risks. For example, treatment with testosterone often leads to masculinization of secondary sex characteristics and hypertrophy of the clitoris with resulting neuropathy. Aside from hormone replacement therapy, medical management of FSD remains in early experimental phases.

Vasoactive substances have been in use for male ED and are under investigation for FSD. Some of these medications include local or topical estrogen application that relieves symptoms of vaginal dryness, burning, and urinary frequency and urgency. Topical testosterone jell may result in similar amelioration with the added benefit of increasing esthesia. In menopausal women, or oophorectomized women, complaints of vaginal irritation, pain or dryness can be relieved with topical estrogen cream. A vaginal estradiol ring is now available that delivers low-dose estrogen locally, which may benefit breast cancer patients and other women unable to take oral or transdermal estrogen. Laan, E., Everaerd W., Physiologic al measures of vaginal vasocongestion. Int. J. Impt. Res. 10: 107-110 (1998). It is contemplated that these treatments may be further enhanced with colpoplasty as disclosed herein.

A popular but extremely cautious treatment for FSD focuses on methyl testosterone (MT). This treatment is often used in combination with estrogen in menopausal women to relieve symptoms of inhibited desire, dyspareunia or hypolubrication. There are conflicting reports regarding the benefit of methyl testosterone and/or testosterone cream for treatment of inhibited desire and/or vaginismus in pre-menopausal women. Potential benefits of this therapy include increased clitoral sensitivity, increased vaginal lubrication, increased libido, and heightened arousal. Potential side effects of testosterone administration, however, are significant, and whether topical or oral, include weight gain, clitoral enlargement, increased facial hair and high cholesterol.

Sildenafil, the medication commonly known as Viagra®, serves to increase relaxation of clitoral and vaginal smooth muscle and blood flow to the genital area. This result, although affecting arousal and lubrication, does not increase desire or esthesia. Goldstein I, Berman J R, Vasculogenic female sexual dysfunction: vaginal engorgement and clitoral erectile insufficiency syndromes. Int. J. Impot. Res. 10: 84-90 (1998). Sildenafil, in combination with other vasoactive substances, may be used for treatment of female sexual arousal disorder. In combination with colpoplasty as described herein, the possibility of arousal and increased esthesia to climax is greatly enhanced.

Clinical studies evaluating safety and efficacy sildenafil in women with SAD are in progress. Several studies are already published demonstrating efficacy of sildenafil for treatment of FSD secondary to Serum Seratonin Reuptake Inhibitor (SSRI) use. Park K, Goldstein I, Andry C, et al: Vasculogenic female sexual dysfunction: The hemodynamic basis for vaginal engrogement insufficiency and clitoral erectile insufficiency. Int. J. Impoten. Res. 9: 27-37 (1988); Nurnberg H G, Lodillo J, Hensley P, et al., Sildenafil for iatrogenic seratonergic antidepressant medication-inducted sexual dysfunction in 4 patients. J. Clin. Psych. 60(1): 33 (1999).

Another medication to be considered in combination with colopoplasy is L-arginine. L-arginine functions as a precursor to the formation of nitric oxide, which mediates relaxation of vascular and non-vascular smooth muscle. L-arginine has not been used in clinical trials in women; however preliminary studies in men have been encouraging.

Phentolamine, (Vasomax®) is currently available in an oral preparation. The drug is an adrenergic (α receptor) blocker resulting in vascular smooth muscle relaxation and increased blood flow to the genital area. It has been studied in male patients for the treatment of ED. A pilot study in menopausal women with FSD showed enhanced vaginal blood flow and improved subjective arousal with the medication.

Considering both medical and psychological disciplines, the ideal approach to FSD is a collaborative effort between therapists and physicians. This should include a complete medical, and psychosocial evaluation, as well as inclusion of the partner or spouse in the evaluation and treatment process. In the non-FSD patient, the medical and psychological aspects of combined treatment are not called for and attention to the GS area may be considered without a multidisciplinary approach. In this respect, a woman and her partner may seek colpoplasty merely for enhanced gratification in the sexual act. In either case, a first step in coloplasty as described herein requires identification of the GS area and the GS proper.

The GS area is hyperesthetic area identified about the anterior vaginal wall. It is usually located about halfway between the posterior aspect of the pubic bone and the cervix, along the midline course of the urethra and near the neck of the bladder. Stimulation of this area often results in rhythmic contracture of the pubococcygeous muscle, which is one of many muscles involved in the vaginally generated orgasm. While the pudendal nerve primarily innervates the clitoris, the GS area accepts nerve fibers from both the pelvic nerve and hypogastric plexus. The GS area includes tissue associated with the periurethral glands and includes the Skenes' glands and Halban's fascia; the counterpart of which in the man is the prostrate gland surrounding the proximal (male) urethra.

Upon palpation, the GS proper may be palpated and is a small area of indurated and, at the same time, spongy tissue. Stimulation causes vasodilation and swelling where an increase in diameter from the 1 cm 3 cm and in some cases a 5 cm diameter will be palpable.

The GS area, then, is located on the anterior wall of the vagina approximately 3-4 cm from the distal urethra on a midline with the urethra extending just proximally to the cystourethral juncture. Clinically, upon stimulation of the GS area, a subject will usually describe a sensation of urinary urgency. This relates to the GS area's anatomical association with the paraurethral glands and urethra.

In their 1981 study, Perry and Whipple hypothesized that the Grafenberg spot was probably composed of a complex network of blood vessels, the paraurethral glands and ducts, nerve endings, and the tissue surrounding the bladder neck, but they did not conduct any anatomical studies in this area.

Despite the evidence that specific anatomical structures correspond to the area defined as the Grafenberg spot, its exact anatomical identity remains inconclusive. This may be due to expected anatomical differences in size, innervation and location among women in general. A recent study by O'Connell in Australia posited that the clitoris was an organ of much the same dimensions and anatomical divisions as the male penis. The study investigated the anatomical relationship between the urethra and the surrounding erectile tissue, and reviewed the appropriateness of the current nomenclature used to describe this anatomy. A detailed dissection was performed on 2 fresh and 8 fixed human female adult cadavers (age range 22 to 88 years). The relationship of the urethra to the surrounding erectile tissue was ascertained in each specimen, and the erectile tissue arrangement were determined and compared to standard anatomical descriptions. Nerves supplying the erectile tissue were carefully preserved and their relationship to the soft tissues and bony pelvis were noted. The study concluded that the female urethra, distal vaginal wall and erectile tissue were packed into the perineum caudal (superficial and inferior) to the pubic arch, which is bounded laterally by the ischiopubic rami, and superficially by the labia minora and majora. This complex was reported not as flat against the rami, as is commonly depicted, but projected from the bony landmarks for 3 to 6 cm. The perineal urethra was noted to be embedded in the anterior vaginal wall and surrounded by erectile tissue in all directions. Although the study attempts to associate its findings with redefining the anatomical clitoris, it is apparent that the findings are consistent with the work of Grafenberg, Whipple, Davidson, Ladas and Perry.

Although the exact anatomical location of the GS area and GS proper vary, the GS area and GS proper are confidently defined by association with the paraurethral and Skenes glands, pubic symphysis, urethra, bladder and vestibule all or which, or combinations of which, serve as appropriate anatomical landmarks for augmentation consistent with the invention described herein. In light of the difficulty in perfecting the exact location of the GS area in a given subject, a specialized speculum is described.

In a first step of the invention, then, the GS area of an adult female subject should be clinically identified and localized via superficial anatomical landmarks and direct guidance by the subjects. In the lithotomy position, the GS area may be located along the midline of the anterior vagina from the front of the inferior aspect of the pubic symphysis to the midpoint of the GS area corresponding to location 3-4 cm distal to the cervix on the anterior wall of the vagina. The linear distance from the distal urethra or vestibule to the midpoint of the GS area should be measured with a non-flexible or mildly flexible measuring device. Measuring devices are well known in the art and certain modifications, discussed below, may assist the surgeon in locating and marking the GS area.

In the alternative, the method for finding the G-Spot in vivo is consultation and self examination by the patient to locate the G-Spot. The clinician digitally palpates the area located by the patient at or about the anterior vagina. The clinician may then palpate the surface anatomy of the patient at or about the mons pubis or symphysis pubis by proprioception of the digit used for GS area identification and the thumb opposed against the antero-inferior surface of the pubic symphysis against the digit used for GS area identification. Opposing digital “contact” is maintained and the GS area identification digit is withdrawn from the GS area and vestibule. At this point, a measurement may be taken of the location of the thumb at or about the pubic symphysis or mons pubis. A surgical marking pen is then use to mark the point of the thumb “contact” to the previous placement of the GS area identification digit and then the measuring ruler is placed at this point and the distal measurement taken. This is the distance from the anterior surface of the pubic symphysis and the GS area. A specially designed speculum is then placed in the vaginal canal and the heel of the speculum is marked where it aligns with the anterior pubic bone. The speculum is then removed and the measuring device is placed at the mark at the heel of the speculum and the prior distance recorded is marked at the distal area at the injection port window of the specially designed speculum. A traditional speculum is appropriate for a similar type of measurement. This distal mark indicates the location of the GS area. The speculum is then reinserted and caution is used to make sure that the mark at the heel is consistent with the anterior pubic symphysis. By doing so the mark at the injection port of the speculum indicates the position of the G-Spot.

In either method, the distance identified with the measuring device should be recorded. Confirmation of this measurement may be accomplished by asking the subject to again locate the GS area and confirming that the distance measured clinically and measured manually by the subject are substantially the same. If a subject is unable to identify the GS area, the surgeon may still be confident in his/her clinical evaluation of the anatomical GS area location.

The material to be used for injection/augmentation may be pre-loaded into a syringe for delivery of the material. In the alternative, certain materials may be loaded into syringe for delivery intra-operatively. In the alternative, permanent materials may be used, some of which are not suited for delivery via a syringe. These materials are discussed in more detail below.

In one preferred embodiment of the invention, collagen is pre-loaded into a syringe. One preferred collagen source may be human allograft material of the fascia lata of the lower extremity collected from human cadaver donors. Donor suitability should be determined by the appropriate tissue bank according to the standards of the American Association of tissue Banks (MTB) and the FDA. Donor serum should be negative by FDA-licensed tests for human immunodeficiency virus antibody (anti-HIV 1-2), Hepatitis B surface antigen (HBsAG), Hepatitis B core antibody (HBcAB), Hepatitis C antibody (anti HCV), human T-lymphotropic virus type I antibody (anti HTLV-1), and rapid plasma reagin (RPR) for syphilis. In this embodiment of the invention, as well as the embodiment of the invention discussed as a kit for colpoplasty, the material should be freeze-dried, irradiated, particulated, and vacuum-sealed.

Certain collagen sources may or may not require subject pre-testing for allergic reaction. Collagen banks, or other tissue banks, may be used for collagen or other tissues used for colpoplastic implantation. For example, tissue for implantation may be obtained from LifeLink Tissue Bank, Tampa, Fla., USA, <http://www.lifelinkfound.org/bank.html>; PathServe Autopsy and Human Tissue Bank San Francisco, Calif., USA, <http://www.tissuebank.com/>; and Peterborough Hospital Human Research Tissue Bank which is based in Peterborough District Hospital, Cambridgeshire, UK, <http://www.tissuebank.co.uk/>, as well as many other suppliers of tissue. Implantation may also be performed by autograft of a subjects own tissue; tissue from liposuction, for example.

The amount used for the augmentation of the GS area may range from at least about 50 mg to 300 mg, 100 mg to 275 mg, 150 to 250 mg and 200 to 230 mg. Further ranges include but are not limited to at least about 140 to 220 mg, 160 to 250 mg, and is preferably at least about 240 mg when using collagen as opposed to other absorbable substances such as poly galactic acid. The collagen may be between 0.5 to 4 mm, 1 to 3 mm and preferably 1-2 mm particulate size. These formulations will deliver easily through a 3 cc to 10 cc leur lock syringe. The collagen may be re-hydrated by attaching it to another 3 cc to 10 cc leur lock syringe via a patent double edge leur lock connector containing 2 cc of local anesthetic containing epinephrine. For example, 1% lidocaine with epinephrine or 0.5% bupivocaine with epinephrine may be used. The mixture should be agitated back and forth numerous times until the collagen is completely re-hydrated. With collagen, this should be performed at lease ten minutes prior to the GS area colpoplasty.

It is preferred that subjects be pre-operatively medicated with of a broad spectrum antibiotic, 100 mg of doxycycline for example, orally or parenterally at least one hour prior to GS area colpoplasty. The antibiotic should be continued a minimum of three days post GS area colpoplasty.

The vaginal canal should be prepped with three separate passes of three separate povodone iodine swabs. Specially designed L-shaped metric rulers are available and may be preset with the subjects GS area measurements in preparation for marking the GS area proper. GS area access can be obtained with a sterile speculum. A traditional speculum is adequate. The inventor has developed a specially designed speculum consisting of a lower blade and a self retaining upper blade capable of bifurcating in the midline giving access to the anterior vaginal wall and unimpeded exposure of the inferior aspect of the anterior portion of the pubic symphysis; this facilitates measurement of the GS area. This specially designed speculum represents the subject matter of a separate application for letters patent both in the United States and abroad. Further, it is contemplated that this specialized speculum will form a part of a kit claimed herein for use in performing colpoplastic procedures disclosed in this invention. Said kit is contemplated to contain either temporary or permanent materials for use in colpoplasty, needles of varying gauge and length, povodone iodine swabs, measuring and labeling materials. Needle lengths in the kit should range from 8 to 15 cm and gauges of needles should range from 10 to 18. Materials for colpoplasty should be consistent with those disclosed herein. Anesthetics may or may not be included, but at least about three to five povodone iodine swabs should be included.

In one preferred embodiment of the invention a temporary colpoplasty may be performed. Said temporary colpoplasty should be performed with the subject in the lithotomy position. A speculum should be used for visualization of the anatomy of the vagina. Lubricant, as with most gynecological procedures, is indicated here. Attention should be directed to exposing the anterior vaginal wall and inferior aspect of the pubic symphysis as described above. The vertical limb of the preset L-shaped measuring device may then be placed against the anterior surface of the pubic symphysis and the horizontal limb introduced into the vagina along the midline of the anterior wall. The intra-vaginal distal end of the measuring device represents the midpoint of the subject's GS area along the midline of the anterior vagina. The GS area or GS proper should then be marked with either a standard surgical marker or a marker specially designed for the procedure.

Infiltration of 1-3 cc of an anesthetic with epinephrine should be injected just deep to the previously marked GS area. Anesthetics appropriate for use are those that result in the appropriate local anesthesia via infiltration of 5 cc or less of anesthetic. For example, 1 or 2% lidocaine, 0.5 or 0.75% bupivocaine may be delivered to the area at a volume less than 5 cc and create the appropriate local blockade or anesthesia. Certain anesthetics, such as cocaine hydrochloride, may be used without epinephrine, as the anesthetic is a hemostatic agent in and of itself. Any anesthetic, either amide based or ester based, may be used. However, due to the longer acting capabilities of the amide anesthetics, amide anesthetics are preferred.

At this point in the procedure, anesthetic, it is noted, is used not only for neural blockade but for submucosal hydrodissection, or fluid adhesiotomy, depending on the condition of the soft tissue. For this purpose a 5 cc leur lock syringe with a 3½ spinal needle may be used. A minimal gauge needle, such as a 30 gauge, 27 gauge and no more than a 25 gauge needle should be used for neural blockade and submucosal hydrodissection. Care should be taken to insert the needle just deep to the submucosa fascial line in the frontal plane. The surgeon should remember to aspirate the syringe just prior to injection to insure against vascular infiltration.

The absorbable material, collagen in this description, may be re-hydrated by connecting one syringe containing a hydrating liquid to another syringe containing the collagen. A syringe connector is usually supplied with the collagen and comprises two leur lock ends on a small cylinder. The connector is used for aseptic transfer or the hydrating liquid to the syringe containing the collagen. Once the liquid has been transferred, the collagen will rehydrate.

The now re-hydrated collagen should be agitated multiple times, both initially and a few more times prior to injection. The leur lock syringe containing the re-hydrated collagen should then be removed from the connector and attached to a needle extender at the end of which a luer lock needle at least about 14 gauge 13.5 cm needle is attached The needle may be at least about 10 to 18 from at least about 8 cm to 15 cm. The gauge of the needle will depend upon the selection of augmentation material to be used; a 14 gauge needle is adequate for delivery of collagen. The extender and needle should be primed with collagen and then directed to the previously marked GS area. Care should be taken to insert the needle parallel to the frontal plane and perpendicular to the transverse plane. Attention should be directed to the submucosa of the GS area along a midsagital line. Again, syringe aspiration should be performed prior to injection of the collagen. The collagen should be injected in a slow and deliberate manner. At least about 240 mg of 1 mm particle size re-hydrated collagen in 2 cc of 1% lidocaine (said lidocaine used as a hydrating liquid) will create an augmented GS area of at least or about 15 to 20 mm in diameter with an anterior vaginal height of at least or about 5-10 mm. The injection site may or may not need a temporary gauze pressure dressing to control bleeding. The procedure is complete after removal of all instrumentation and after all bleeding has subsided. The subject will usually be able to verify correct collagen placement upon normalized esthesia following diminished anesthesia.

In the above description, collagen is used as a material of choice for colpoplasty. However, other materials that may be used for temporary colpoplasty can be selected from the group consisting of fibril, adipose, alloderm, cymetra, and fascian. These materials may form a part of a kit for colpoplasty disclosed herein.

In another embodiment of the invention, the colpoplsty may be permanent. In this case, a non-absorbable material is used in the injection. Non-absorbable materials may be selected from the group consisting of silicon, silastic beads, e-PTFE, polyacylamide gel, and calcium hydroxiapatite. Other permanent materials include, but are not limited to, ceramics, surgical and stainless steals, glass, polymethyl-methacrylate and other plastics. It is contemplated that these permanent materials may be selected and used in anatomically compatible shapes such as oblong, round, oval, linear, cuboidal and the like. Permanent implants may be sized by displaceable volume, i.e., a space filling implant. Volumes may be used that range from at least about 1 cc to 10 cc, 2-9 cc, 3 to 7 cc, 4 to 6 cc. A volume of at least about 3 to 5 cc is preferred or that volume that creates an augmented GS area of at least about 15-20 mm in diameter and 5-10 mm in height. Said materials may also form part of a kit for colpoplasty disclosed herein. .

In embodiments of the invention not suitable for injection the permanent implants disclosed herein may be placed in the GS area using other surgical technique. Following the same preparation of the subject, identification and marking of the GS area and local anesthesia, this procedure would further involve a small, between 2 and 15 mm, incision in an area just caudal to the GS area. The GS area location should be pre-measured and marked as described above. For this incision, a No. 15 blade or Beaver-type blade may be used. All techniques and protocols for minimal incision surgery, as is known in the art, should be followed. The incision should be deepened no more than that necessary to accommodate the chosen implant. Soft tissue dissection technique should be used to create a pocket for nesting of the implant. Once the pocket is created, the implant may be nested. The implant may be selected from the group consisting of glass, silicone, stainless steel, surgical plastics e-PTFE, ceramics and other inert materials designed for implantation in humans. Once placement of the implant is perfected, the area should be copiously flushed with an antibiotic solution. Absorbable suture, such as poly galactic acid suture, should be used to close the surgical site. A short-term pressure dressing over the surgical area may be used but may not be required.

The foregoing descriptions of embodiments of the invention are intended to be illustrative of the embodiments of at least one aspect of the invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein. All publications and patent applications are incorporated by reference as if fully set forth herein. At least one aspect of the invention is further illustrated by the following examples, which are not intended to limit the effective scope of the claims.

### EXAMPLE I

Colpoplasty in an Anorgasmic Subject.

A patient presented with a complaint of long term inability to achieve orgasm. Further, patient complained of hypolubrication and. low sexual desire and arousal. A history and physical examination revealed a well nourished female above the age of consent and in no acute distress. Allergies and significant past medical history were denied and reported as negative for complications. The medical practitioner advised a colpoplasty via augmentation of the Grafenberg spot area using collagen obtained from an appropriate tissue bank. Colpoplasty was performed as follows.

240 mg of collagen as was chosen to create a 15-20 mm diameter and 5-10 cm high augmentation of the Grafenberg spot area. The collagen was of a 1 mm particulate size. Collagen was re-hydrated by attaching the collagen containing syringe to another leur lock syringe of similar volume capacity via a patent double edge leur lock connector containing 2 cc of 1% lidocaine with epinephrine. The mixture was agitated back and forth numerous times until the collagen was completely re-hydrated. Agitation and re-hydration occurred at lease ten minutes prior to the colpoplasty.

The patient was pre-operatively medicated with an oral dose of 100 mg of doxycycline, one hour prior to colpoplasty.

With the patient in the lithotomy position, the vaginal canal was be prepped with three povodone iodine swabs. Specially designed L-shaped metric rulers were selected to be preset with the patient's GS area measurements in preparation for marking the GS area. GS area access was obtained with a sterile speculum with removably attachable light source. Lubrication was used as necessary throughout the colpoplasty.

Attention was then directed to exposing the anterior vaginal wall and the palpable inferior aspect of the pubic symphysis. The vertical limb of the preset L-shaped measuring device was then placed against the anterior surface of the pubic symphysis and the horizontal limb introduced into the vagina along the midline of the anterior wall. The intra-vaginal distal end of the measuring device represented the midpoint of the patient's GS area along the midline of the anterior vagina. The GS area was then marked with a standard surgical marker.

Infiltration of 3 cc of an anesthetic with epinephrine was injected just deep to the previously marked GS area. Neural blockade and submucosal hydrodissection was accomplished. For this purpose a 5 cc leur lock syringe with a 3½ spinal needle (27 gauge) was used. Care was taken to insert the needle just deep to the submucosal fascial line in the frontal plane. The surgeon aspirated the syringe just prior to injection to insure non-vascular invasion.

The collagen was hydrated consistent with the disclosed invention. The re-hydrated collagen was agitated appropriately prior to injection. The leur lock syringe containing the collagen was removed from the connector and attached to a needle extender at the end of which a luer lock 14 gauge 13.5 cm needle was attached. The extender and needle was primed with collagen and then directed to the previously marked GS area. Care was taken to insert the needle parallel to the frontal plane and perpendicular to the transverse plane. Attention was directed to the submucosa of the GS area along a midsagital line. Syringe aspiration was again performed prior to injection of the collagen. The collagen was injected in a slow and deliberate manner. 240 mg of 1 mm particle size re-hydrated collagen in 2 cc of 1% lidocaine created an augmented GS area of at least or about 20 mm in diameter with an anterior vaginal height at least or about 10 mm. The injection site needed only slight pressure with a “peanut” gauze for 45 seconds. The procedure was complete after removal of all instrumentation and after all bleeding had subsided. The patient was easily able to verify correct collagen placement once anesthesia had subsided.

Upon a return visit to the surgeon's office, the patient reported markedly diminished FSD symptoms and an ability to achieve multiple climactic events during sexual relations.

### EXAMPLE II

Study of FSD Women and Normally Sexually Functioning Women before and after Colpoplasty

The Grafenberg spot area of 15 adult, pre-menstrual, sexually experienced female subjects above the age of consent were clinically identified and localized via direct guidance by the female subjects. In the lithotomy position, the subjects measured the distance of the GS area along the midline of the anterior vagina from the front of the inferior aspect of the pubic symphysis to the midpoint of the GS area. This was accomplished by using the middle finger of the dominant hand in a rectus fashion, placing the tip of the finger at the center of the GS area and using the thumb to mark the spot at the distal portion of the finger against the pubic symphysis. The distance for each subject was measured and recorded using a metric ruler. The researcher confirmed the location of the GS area and confirmed that the researcher's measurements were substantially similar to the subject's localization. The GS area measurement was used to determine the injection point into the GS area.

The material selected for GS augmentation was collagen from human allograft material, specifically fascia lata, collected from human cadaver donors (name of tissue bank). Donor suitability was determined by the tissue bank according to the standards of the American Association of Tissue Banks (MTB) and FDA. Donor serum was negative by FDA-licensed tests for human immunodeficiency virus antibody (anti-HIV 1-2), Hepatitis B surface antigen (HBsAG), Hepatitis B core antibody (HBcAB), Hepatitis C antibody (anti HCV), human T-lymphotropic virus type I antibody (anti HTLV-1), and rapid plasma reagin (RPR) for syphilis. The material had been freeze-dried, irradiated, particulated, and vacuum-sealed.

The collagen source did not require subject pre-testing for allergic reaction. The amount used for the augmentation of the GS area was 240 mg of 1 mm particulate size which was contained in a 3 cc leur lock syringe. The collagen was re-hydrated by attaching it to another 3 cc leur lock syringe via a patent double edge leur lock connector containing 2 cc of 1% lidocaine. The mixture was agitated back and forth numerous times until the collagen was completely re-hydrated. This was performed for each subject at lease ten minutes prior to the G-Spot augmentation.

All subjects were pre-medicated with 100 mg of doxycycline orally one hour before collagen injection. (This dose of doxycycline was continued every twelve hours for three doses post-injection.) The vaginal canal was prepared with three povodone iodine swabs. A specially designed L-shaped metric ruler was preset with each individual subject's GS area measurements in preparation for marking. GS area access was obtained with a sterile, specially designed speculum. The specially designed speculum consisted of a modified Welsh Allen speculum wherein an upper dilatory blade is modified by removing an amount of material, plastic for example, in a fashion suitable for delivering soft tissue though the said removed plastic to better expose the soft tissue and frame the area to be addressed.

The subjects were placed in the lithotomy position and the speculum was introduced into each subject's vagina using a small amount of lubricating jelly and thereby exposing the anterior vaginal wall and inferior aspect of the pubic symphysis. The vertical limb of the preset L-shaped measuring device was placed against the anterior surface of the pubic symphysis and the horizontal limb was introduced into the vagina along the midline of the anterior wall. The intra-vaginal distal end of the measuring device represented the midpoint of each subject's GS area along the midline of the anterior vagina. This location was marked with a surgical marker. After the measuring device was removed, 3 cc of 1% lidocaine with epinephrine was injected just deep to the GS area (the vaginal submucosal) in each subject for anesthesia and hydro-dissection. For this purpose a 5 cc leur lock syringe with a 3½″ spinal needle was used. Care was taken to insert the needle just beneath the submucosa in a horizontal fashion and to aspirate prior to injection to insure non-vascular invasion.

The re-hydrated collagen was re-agitated appropriately prior to each injection. The 3 cc leur lock syringe containing the collagen was removed from the connector and attached to a needle extender at the end of which a 14 gauge 13.5 cm needle was attached. The extender and needle were primed with collagen in each case and then directed to the marked GS area. Care was taken to insert the needle parallel to the frontal plane and just deep to the submucosa of the GS area at its midpoint (previously marked). Syringe aspiration was performed prior to injection. Slow injection of collagen was performed. 240 of the 1 mm particle size re-hydrated in 2 cc of 1% lidocaine created an augmented GS area averaging among the subjects of at least about 15 to 20 mm in diameter with a vaginal height of at least about 5 to 10 mm in each subject.

In follow up, no complications were seen in any subject. Each subject reported enhanced sexual responsiveness, desire and ability to achieve orgasm.

The female vaginal sexual arousal and esthesia were investigated in normal sexually functioning pre-menopausal women and FSD women with varying symptomotolgy including, but not limited to, dysesthesia, dyserethism, hypolubrication and other symptoms consistent with FSD. The data are outlined below for a total of 15 women who participated in the study. Pre-injection and post-injection self-reports containing a number of questions specific to the colpoplasty were taken to access the sexual responsiveness and general esthesia of the subjects before and after the colpoplasty. The results for pre-injection and post-injection surveys were analyzed using the Student's t-test and a p<0.05 was taken as significant.

The averaged results of aspects of the study pertinent to the invention disclosed herein are included in Table 1. Of the participants studied, ability to reach climax after colpoplasty was significantly increased (p<0.05). Moreover, the time taken to reach orgasm was significantly decreased after colpoplasty (p<0.01). Overall, the subjects that reported that they achieved climax via GS area stimulation measured about 27% before colpoplasty. Subjects who were either unsure or who reported no GS area climax were nearly 73%. After colpoplasty, only 20% of subjects were unsure or reported an inability to reach vaginal or GS area climax while 80% reported an ability to reach GS area climax. This represents a nearly threefold increase in ability to reach GS area climax in post colpoplasty versus pre colpoplasty subjects.

A profound improvement was observed for subjects reaching GS area climax during sexual activity with a partner. Therefore, based on the above results, this example establishes, at a minimum, that colpoplasty significantly improves female sexual esthesia and may form an important role in normal and abnormal female sexual response and esthesia.

TABLE 1

| | |
|---|---|
| PRE-INJECTION | POST-INJECTION |

During sexual activity of any type,
how often does the subject reach climax

| | |
|---|---|
| 46.7% | 61.3%. |

How often, during self-stimulation, does the subject reach climax

| | |
|---|---|
| 62.7% | 69.3% |

How often during sexual intercourse does the subject reach climax

| | |
|---|---|
| 50.7% | 82.7% |

Time (min.) for does the subject to reach climax via clitoral stimulation

| | |
|---|---|
| 16.3 min | 10.0 min. |

Was subject ever able to reach climax through stimulation of GS area

| | |
|---|---|
| (33.3%/no) (40.0%/unsure) | (13.3%/no) (6.7%/unsure) |
| (26.7%/yes) | (80%/yes) |

If able to reach climax via GS area stimulation,
how long did it take to reach climax

| | |
|---|---|
| 17 min.* | 8 min.* |

*Only subjects capable of offering a time for GS area climax are included in the average. One subject was unable to climax after injection without being able to climax prior to injection. Six subjects who admitted GS area anorgasmia were able to reach GS area climax after injection.

All references cited herein are incorporated by reference in their entirety. The descriptions in the present invention are provided only as examples and should not be understood to be limiting on the claims.

1. A method of colpoplasty comprising:
localization of a Grafenberg spot area in a vagina;
anesthetizing the localized Grafenberg spot area; and
augmenting the anesthetized Grafenberg spot area.

2. The method of claim 1 wherein the Grafenberg spot area is located on an anterior wall of the vagina.

3. The method of claim 2 wherein the Grafenberg spot is further located on the anterior wall of the vagina between a vestibule and a cervix.

4. The method of claim 3 wherein the Grafenberg spot is further located at an area between the vestibule and the cervix at an area at or 1 to 5 cm proximal to a pubic symphysis to an area at or 1 to 5 cm distal to the cervix.

5. The method of claim 2 wherein the Grafenberg spot is located substantially along the urethra.

6. The method of claim 1 wherein the augmentation is permanent or temporary.

7. The method of claim 6 wherein permanent augmentation comprises implantation deep to the Grafenberg spot area of a volume of an non-absorbable material selected from the group consisting of silicon, e-PTFE, polyacylamide gel, calcium hydroxyapatite, polymethyl-methacrolate, silastic beads, surgical steel, stainless steel, glass, ceramics and plastics.

8. The method of claim 7 wherein the volume of non-absorbable material is at least about 1 cc to 10 cc.

9. The method of claim 7 wherein the volume of non-absorbable material is at least about 2 cc to 9 cc.

10. The method of claim 7 wherein the volume of non-absorbable material is at least about 3 cc to 7 cc.

11. The method of claim 7 wherein the volume of non-absorbable material is at least about 4 cc to 6 cc.

12. The method of claim 7 wherein the volume of non-absorbable material is 3 to 5 cc.

13. The method of claim 6 wherein the temporary augmentation comprises implantation deep to the Grafenberg spot area an amount of an absorbable material selected from the group consisting of collagen, fibril, adipose, alloderm, cymetra, fascian and poly galactic acid.

14. The method of claim 13 wherein the amount of absorbable materials is at least about 100 mg to 300 mg.

15. The method of claim 13 wherein the amount of absorbable materials is at least about 100 to 275 mg.

16. The method of claim 13 wherein the amount of absorbable materials is at least about 150 to 250 mg

17. The method of claim 13 wherein the amount of absorbable materials is at least about 200 to 230 mg.

18. The method of claim 13 wherein the absorbable material is collagen.

19. The method of claim 17 wherein the collagen is implanted in an amount s of least about 140 to 220 mg.

20. The method of claim 17 wherein the collagen is implanted in an amount least about 160 to 250 mg.

21. The method of claim 19 wherein the amount of collagen is at least about 240 mg.

22. The method of claim 17 wherein the collagen is of a particulate size of at least about 0.5 to 3 mm.

23. The method of claim 22 wherein the particulate size is at least about 1-2 mm.

24. A method for treating female sexual dysfunction comprising a chemotherapy in combination with localization of a Grafenberg spot area in a vagina; anesthetizing the localized Grafenberg spot area; augmenting the anesthetized Grafenberg spot are in combination with use of a chemotherapy.

25. The method of claim 24 wherein the female sexual dysfunction is selected from a group consisting of sexual pain disorders, hypolubrication, anorgasmic disorders, sexual aversion disorder, hypoactive sexual desire disorder, iatrogenic and drug related sexual disorders.

26. The method of claim 24 wherein the chemotherapy is selected from a group consisting of hormones, anti-depressants, amino acids, muscle relaxants, sildnenefil, and aphrodisiac.

27. A method of treating female sexual dysfunction comprising; psychotherapy in combination with localization of a Grafenberg spot area in a vagina; anesthetizing the localized Grafenberg spot area; augmenting the anesthetized Grafenberg spot are in combination with use

28. The method of claim 27 wherein the psychotherapy may be selected from a group consisting of psychiatry, psychology marriage counseling, family counseling, and relationship counseling.

29. A kit for performing a temporary colpoplasty comprising: a needle; a syringe; an amount of an absorbable material; a speculum; and instructions for using the kit.

30. The kit of claim 29 further comprising a measuring device.

31. The kit of claim 29 further comprising a light source wherein said light source is removably attachable to the speculum.

32. The kit of claim 29 further comprising syringe connector comprising a first luer lock and a second leur lock wherein said luer locks are on opposite sides of the syringe connector.

33. The kit of claim 29 further comprising a surgical marking device.

34. The kit of claim 29 further comprising a plurality of povodone iodine swabs.

35. The kit of claim 29 wherein the absorbable material is selected from a group consisting of collagen, fibril, adipose, alloderm, cymetra, fascian and poly galactic acid.

36. The kit of claim 29 wherein the absorbable material is collagen

37. The kit of claim 36 wherein the collagen is supplied in an amount selected from a group consisting of at least about 140 to 220 mg or

38. The kit of claim 36 wherein the collagen is supplied in an amount of about 160 to 250 mg.

39. The kit of claim 37 wherein the collagen is supplied in an amount of 240 mg.

40. The kit of claim 37 wherein the collagen is of a particulate size of at least about 0.5-3 mm.

41. The kit of claim 39 wherein the collagen is of a particulate size of at least about 1-2 mm.

42. The kit of claim 29 wherein the needle is at least about 10-18 gauge.

43. The kit of claim 42 wherein the needle is at least about 14 gauge.

44. The kit of claim 29 wherein the needle is at least about 8 cm to 15.

45. The kit of claim 44 wherein the needle is at least about 13.5 cm.

46. The kit of claim 29 further comprising a needle extender.

47. A kit for permanent colpoplasty comprising: a speculum; an amount of a non-absorbable material; and instructions for using the kit.

48. The kit of claim 47 further comprising a beaver blade and a No. 15 blade.

49. The kit of claim 47 further comprising an absorbable suture material.

50. The kit of claim 47 further comprising a light source wherein said light source is removably attachable to the speculum.

51. The kit of claim 47 further comprising a plurality of povodone iodine swabs.

52. The kit of claim 47 further comprising a measuring device.

53. The kit of claim 47 further comprising a surgical measuring device.

54. The kit of claim 47 wherein the non-absorbable material is selected from a group consisting of silicon, e-PTFE, polyacylamide gel, calcium hydroxyapatite, polymethyl-methacrolate, silastic beads, surgical steel, stainless steel, glass, ceramics and plastics.

55. The kit of claim 47 wherein the volume of non-absorbable material is of at least about 1 cc to 10 cc.

56. The kit of claim 47 wherein the volume of non-absorbable material is at least about 2 cc 9 cc,

57. The kit of claim 47 wherein the volume of non-absorbable material is at least about 3 cc to 7 cc

58. The kit of claim 47 wherein the volume of non-absorbable material is at least about 4 cc to 6 cc.

59. The kit of claim 47 wherein the volume of non-absorbable material is at least about 3 cc to 5 cc.
